(19) United States
(12) Patent Application Publication
(10) Pub. No.: US 2010/0132055 A1
(43) Pub. Date: May 27, 2010

(54) ANIMAL MODEL FOR THE SELECTION AND VALIDATION OF AGENTS ACTIVE AGAINST PULMONARY EMPHYSEMA AND COLORECTAL CANCER

(75) Inventors: Harald von Melchner, Kronberg (DE); Frank Wempe, Muhlheim Main (DE); Silke De-Zolt, Rodenbach (DE)

Correspondence Address: Briscoe, Kurt G.;Norris McLaughlin & Marcus, PA, 875 Third Avenue, 8th Floor, New York, NY, 10022, US

(73) Assignee: FRANKGEN BIOTECHNOLOGIE AG, Kronberg (DE)

(21) Appl. No.: 12/441,235

(22) PCT Filed: Sep. 4, 2007
(86) PCT No.: PCT/EP07/59227

§ 371 Date: Mar. 13, 2009

(30) Foreign Application Priority Data
Sep. 15, 2006 (EP) 06120714.8

(51) Int. Cl. G01N 33/48; C12Q 1/68; C12Q 1/02; C12N 5/00; A01K 67/00

(52) U.S. Cl. 800/3; 435/6; 435/29; 435/325; 800/8; 800/9; 800/10; 800/13; 800/21

(57) ABSTRACT

The present invention relates to a non-human animal model, cell and tissue cultures derived therefrom, which do not produce or produce only suboptimal levels of one or more functional sestrins and in addition do not produce or do only produce suboptimal levels of latent transforming growth factor β binding protein 4 (ltbp4). Furthermore, the present invention relates to a method for selecting agents for treating the pulmonary emphysema and/or the colorectal cancer exhibited by utilizing the animal model, cell or tissue culture of the invention. The animal model, cell or tissue culture is suitable for preclinical testing of efficacy, toxicity and bioavailability of potential agents.

The present invention relates to a non-human animal model, cell and tissue cultures derived therefrom, which do not produce or produce only suboptimal levels of one or more functional sestrins and in addition do not produce or do only produce suboptimal levels of latent transforming growth factor β binding protein 4 (ltbp4). Furthermore, the present invention relates to a method for selecting agents for treating the pulmonary emphysema and/or the colorectal cancer exhibited by utilizing the animal model, cell or tissue culture of the invention. The animal model, cell or tissue culture is suitable for preclinical testing of efficacy, toxicity and bioavailability of potential agents.

### BACKGROUND OF THE INVENTION

Chronic obstructive lung disease with pulmonary emphysema (COPD) is a highly prevalent disease that has a large impact on quality of life for patients and their families and kills millions of people worldwide. While the major risk factor for COPD is the inhalation of toxic gases and particles that are primarily but not exclusively generated in tobacco smoke, primary (genetic) airway abnormalities are also involved as only a fraction of smokers develop COPD. COPD is associated with major healthcare costs, largely due to expensive treatments such as long-term oxygen therapy and hospital admissions, as well as indirect costs including loss of working capacity. Moreover, treatment of COPD is limited to symptoms as no pharmacologic intervention has been shown so far to modify the natural history of the disease (Vestbo J. and Hogg, J. C. Thorax. 61:86-8, 2006; Fabbri L. M., et al. Am J Respir Crit Care Med. 173:1056-65, 2006).

Colorectal cancer is one of the most common neoplasms in the Western hemisphere and represents a major public health challenge despite progress in detection and therapy. The overall mortality at 5 years is about 40%. While early stage disease can be cured by surgery, only palliative treatment is available for patients with non-resectable, metastatic cancer whose survival rate is between 7-24 months (Shaheen N. J., et al. Am J. Gastroenterol. 2006).

From the above in becomes apparent that new pharmaceutical compounds for the treatment of both conditions are highly desirable and could be identified with the animal model of the invention, which exhibits both pulmonary emphysema and colorectal cancer as a result of loss of function mutations in the sestrin 2- and the latent transforming growth factor beta binding protein 4 genes.

Transforming growth factor-βs (TGF-βs) belong to a protein superfamily whose members control cell growth and differentiation in a variety of adult tissues and are involved in a wide range of immune and inflammatory responses (Shi Y. and Massague, J. Cell. 113:685-700, 2003; Sheppard D. Proc Am Thorac Soc. 3:413-7, 2006). Most cells secrete TGF-β as a functionally inactive, latent cytokine complexed to its latency associated propeptide (LAP), which interacts with its receptors only after activation by proteolytic cleavage. There are two known, structurally different latent complexes of TGF-β. The small latent complex consists of a mature TGF-β dimer bound to the N-terminal portion of the TGF-β-LAP propeptide. The large latent complex contains, in addition to TGF-β-LAP, one of the three latent TGF-β binding proteins (ltbps 1, 3 and 4). Unless displaced from the ltbps after secretion, TGF-β is usually deposited in the extracellular matrix (ECM) as a high molecular weight complex with ltbp and LAP (Annes J. P., et al. J Cell Sci. 116:217-24, 2003). All 4 existing isoforms of ltbp (ltbp 1-4) have been mutationally inactivated in the mouse (Dabovic B., et al. J Cell Biol. 56:227-232, 2002; Sterner-Kock A., et al. Genes Dev. 16:2264-2273, 2002; Shipley J. M., et al. Mol Cell Biol. 20:4879-87, 2000). While the phenotypes of the knock out mice varied among isoforms, both ltbp3 and ltbp4 mutants develop pulmonary emphysema due to defects in TGF-β signaling. However, in contrast to the emphysema developing mutant ltbp3 mice, which is relatively mild and strictly developmental (Dabovic B., et al. J Cell Biol. 56:227-232, 2002), emphysemas in mutant ltbp4 (ltbp4^{-/-}) mice worsen with age and progressively acquire the features of late onset emphysemas that develop in the context of COPD in humans (Sterner-Kock A., et al. Genes Dev. 16:2264-2273, 2002; WO 03/015505). The potential relevance of ltbp4 in the pathogenesis of COPD has been recently underscored by a clinical study reporting a highly significant correlation between specific COPD symptoms and a single nucleotide polymorphism (SNP) in ltbp4 (Hersh C. P., et al. Am J Respir Crit Care Med. 173:977-84, 2006). In addition to the emphysema, mutant ltbp4 mice develop invasive colorectal cancer (Sterner-Kock A., et al. Genes Dev. 16:2264-2273, 2002; WO 03/015505). A link between ltbp4 expression and cancer progression has also been recently reported in a clinical study of patients with colon cancer (Bertucci F., et al. Oncogene. 23:1377-91, 2004).

Sestrin 2 belongs to a family of highly conserved proteins initially discovered as p53 inducible proteins (Velasco-Miguel S., et al. Oncogene. 18:127-37, 1999; Peeters H., et al. Hum Genet. 112:573-80, 2003; Budanov A. V., et al. Oncogene. 21:6017-31, 2002; WO 00/12525; US 2002/0103353). Mammalian cells express three isoforms referred to as sestrin 1 (sestrin 1; also known as PA26), sestrin 2 (sestrin 2; also known as Hi95) and sestrin 3 (FIG. 1). Two of the sestrins (sestrin 1 and sestrin 2) have been recently shown to regulate intracellular peroxide (ROS) levels (Budanov A. V., et al. Science. 304:596-600, 2004; Sablina A. A., et al. Nat Med. 11:1306-13, 2005). More specifically, sestrins are believed to regenerate (reduce) peroxiredoxins (Prxs), which are highly conserved and ubiquitously expressed, antioxidant proteins (reviewed in Georgiou G. and Masip, L. Science. 300:592-4, 2003; Wood Z. A., et al. Trends Biochem Sci. 28:32-40, 2003). While reducing H₂O₂ to H₂O, Prxs are oxidized at their so called peroxidatic cystein (Cys-SH), which is converted into sulfenic acid (Cys-SOH) (FIG. 2). In bacteria, where the Prx AhpC is the primary H₂O₂ scavenger, the oxidized AhpC is subsequently reduced by the dedicated reductase AhpF. However, unlike AhpC, mammalian Prxs are highly susceptible to overoxidation yielding sulfinic acid (Cys-SO₂H) in presence of high peroxide concentrations (FIG. 2). Protein sulfinic acids cannot be reduced by typical cellular reductants such as glutathione and thioredoxin and therefore their formation has been considered an irreversible process. Once Prx has been converted to sulfinic acid it is no longer enzymatically active and inactive enzyme accumulates in cells exposed to oxidative stress (Wood Z. A., et al. Trends Biochem Sci. 28:32-40, 2003). However, more recently gradual recovery of Prxs has been observed after initial oxidative inactivation in eukaryotic cells (Mitsumoto A., et al. Free Radic Biol Med. 30:625-35, 2001; Woo H. A., et al. Science. 300:653-6, 2003) and sestrins have been proposed as the catalyzing enzymes due to their homology to the bacterial sestrin homolog -AhpD-, which regenerates the AhpC Prx and due to their ability to reduce intracellular ROS levels (Budanov A. V., et al. Science. 304:596-600, 2004).

In eukaryotic cells Prxs are believed to be both antioxidants protecting against stress and regulators of ROS-mediated signaling (Wood Z. A., et al. Science. 300:650-3, 2003) It has been known for some time that receptor/ligand interactions generate bursts of ROS, which act as second messengers in signal transduction pathways, including the TGF-β pathway (Bae Y. S., et al. J Biol Chem. 272:217-21, 1997; Lo Y. Y. and Cruz, T. F. J Biol Chem. 270:11727-30, 1995; Mills E. M., et al. J Biol Chem. 273:22165-8, 1998; Sundaresan M., et al. Science. 270:296-9, 1995; Thannickal V. J., et al. J Biol Chem. 273:23611-5, 1998; Thannickal V. J., et al. Faseb J. 14:1741-8, 2000). Defective TGF-β signaling has been shown to be involved in the pathology of pulmonary emphysema and colorectal cancer in both animal models (including WO 03/015505 A3) and men (Morris D. G., et al. Nature. 422:169-73, 2003; Neptune E. R., et al. Nat Genet. 33:407-11, 2003; Sterner-Kock A., et al. Genes Dev. 16:2264-2273, 2002; Massague J., et al. Cell. 103:295-309, 2000; Zhu Y., et al. Cell. 94:703-14, 1998).

The association between ROS and TGF-β is complex and occurs at different levels of the signaling cascade. First, ROS have been shown to activate latent TGF-β1 both in vitro and in vivo (Annes J. P., et al. J Cell Sci. 116:217-24, 2003; Barcellos-Hoff M. H. and Dix, T. A. Mol Endocrinol. 10:1077-83, 1996). In line with this, Fatma et al. have recently reported that lens epithelial cells derived from Prx6 knock out mice are highly susceptible to oxidative stress and develop a phenotype indistinguishable from TGF-β stimulation. They could attribute this phenotype to a ROS mediated activation of latent TGF-β, which was readily reversible by antioxidants (Fatma N., et al. Cell Death Differ. 12:734-50, 2005). Second, TGF-β requires ROS for the induction of various target genes such as PAI-1, CTGF and extracellular matrix genes (Jiang Z., et al. Biochem Biophys Res Commun. 309:961-6, 2003; Park S. K., et al. Biochem Biophys Res Commun. 284:966-71, 2001). Recent studies have shown that TGF-β induced smad2,3 phosphorylation is also partly dependent on ROS (Cucoranu I., et al. Circ Res. 97:900-7, 2005) presumably via the inactivation of a dedicated phosphatase. It has been known for some time that phosphatases are susceptible to inactivation by ROS (Seo J. H., et al. Mol Biol Cell. 16:348-57, 2005; Chiarugi P. and Cirri, P. Trends Biochem Sci. 28:509-14, 2003) and a smad2,3 phosphatase (PPM1A) capable of terminating TGF-β signaling has been recently discovered (Lin X., et al. Cell. 125:915-28, 2006. Third, TGF-β itself induces superoxide production by activating NADPH oxidases presumably by the transcriptional upregulation of Nox4 (Sturrock A., et al. Am J Physiol Lung Cell Mol Physiol. 290:L661-L673, 2006).

### DESCRIPTION OF THE INVENTION

It was now found that the genetic ablation of a sestrin, notably sestrin 2, from the animal model (ltbp4^{-/-} mice model) described in WO 03/015505 resulted in a significant recovery of the disease conditions (see examples), indicating that this family of proteins could provide molecular targets for the treatment of COPD and colorectal cancer. It is believed that ablation of sestrin 2 in the animal model of WO 03/015505, reactivated TGF-β signaling, presumably by increasing intracellular second messenger ROS levels. This in turn significantly improved both disease conditions as illustrated by the examples, suggesting that pulmonary emphysema and colon cancer can be treated by antagonizing sestrin function. The present invention thus relates to

(1) a non-human animal model, which does not produce one or more functional sestrins or produces subotpimal levels of one or more sestrins, preferably sestrin 2 and in addition does not produce latent transforming growth factor β binding protein 4 (hereinafter “ltbp4”) or produces subotpimal levels of ltbp4;
(2) a non-human animal model, which does not express one or more functional sestrins or expresses subotpimal levels of one or more sestrins;
(3) a cell or tissue culture isolated from the animal models as defined in (1) or (2) above;
(4) a method for preparing the non-human animal model of (1) above, which comprises disrupting the sestrin and/or ltbp4 gene in a germ cell of a starting non-human animal;
(5) a method for selecting an agent for treating a symptom occurring in the non-human animal model of (1) above comprising:
(i) applying one or more agents to be tested to said animal model,
(ii) determining whether one or more symptoms occurring in said animal model have changed as a result of application of said agent(s);
(6) a method selecting an agent that interferes with ROS production and TGF-β signaling comprising:
(i) applying one or more agents to be tested to the to the cell or tissue culture of (3) above,
(ii) determining whether cellular ROS levels and TGF-β signaling have changed as a result of the application of said agents or agents;
(7) a method to analyze whether cancer and/or pulmonary emphysema is caused by differential ltbp4 and sestrin gene or protein expression or expression level or by a defect in the ltbp4 and sestrin gene comprising:
(i) characterizing the ltbp4 and sestrin gene or protein expression or expression level or ltbp4 and sestrin gene allele status of an individual having cancer or pulmonary emphysema,
(ii) characterizing the ltbp4 and sestrin gene or protein expression or expression level or ltbp4 and sestrin gene allele status of a control individual, a difference in the ltbp4 and sestrin gene or protein expression or expression level or ltbp4 and sestrin gene allele status indicating that cancer and/or pulmonary emphysema and/or cardiomyopathy is linked to differential ltbp4 and sestrin gene or protein expression or expression level or a defect in the ltbp4 and sestrin genes;
(8) a method for diagnosing cancer and/or pulmonary emphysema and/or cardiomyopathy comprising:
(i) characterizing the ltbp4 and sestrin gene or protein expression or expression level or ltbp4 and sestrin gene allele status of an individual,
(ii) characterizing the ltbp4 and sestrin gene or protein expression or expression level or ltbp4 and sestrin gene allele status of a control individual, a difference in the LTBP-4 gene or protein expression or expression level or ltbp4 and sestrin gene allele status indicating the presence of cancer and/or pulmonary emphysema and/or cardiomyopathy in said individual;
(9) a kit for diagnosing pulmonary emphysema and/or cancer comprising means to detect the ltbp4 and sestrin gene or protein expression or expression level or the ltbp4 and sestrin gene allele status of an individual and a control individual; and
(10) a kit for performing the method of (6) above, said kit comprising the cell or tissue culture of (3) above.

The kits of (9) and (10) above may further contain instructions for performing the methods, in particular for detecting the ltbp4 and sestrin gene or protein expression or expression level or ltbp4 and sestrin gene allele status.

In conclusion, the present invention provides an animal model of human disease that reveals some crucial functions of the sestrin proteins. In view of these functions, the animal model of the invention can be used to develop novel treatments for pulmonary emphysema and cancer that target sestrin proteins and their role in ROS metabolism and TGF-β signaling.

### DESCRIPTION OF THE FIGURES

FIG. 1: Amino acid sequence of the mouse sestrins and homology between isoforms. The sequences of the isoforms are further shown in SEQ ID NOs:1 to 3.

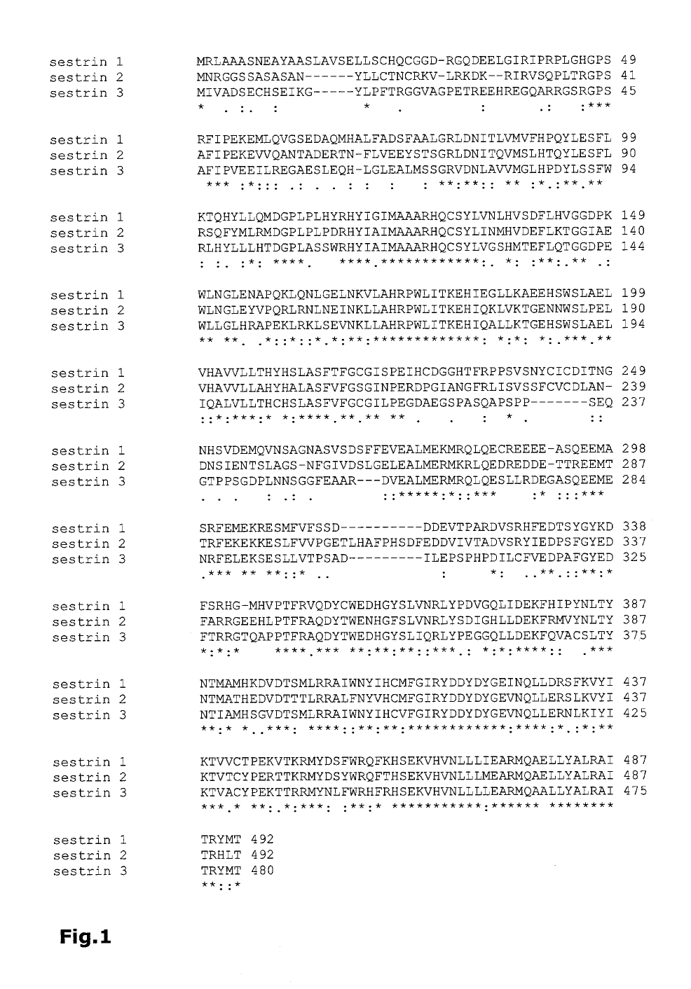

FIG. 2: Oxidation of the peroxidatic cystein of peroxiredoxins (Prxs) by superoxides. Reduced Prxs form dimers via a disulfide bond (1). Oxidized Prxs form sulfenic acid (2), which is reduced by thioredoxin or glutathion in a reverse reaction (1). Overoxidized Prxs form sulfinic acid, which cannot be reduced by thioredoxin or glutathion (3). Sestrins are believed to regenerate the overoxidized Prxs. For further explanation see text.

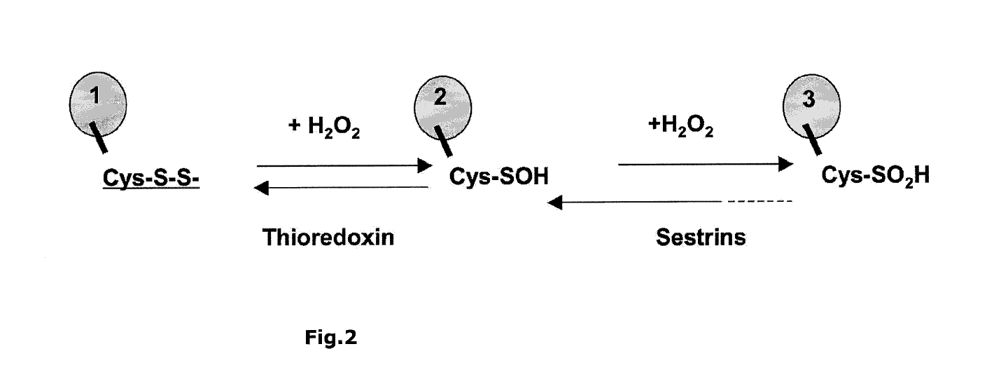

FIG. 3: pT1βgeo gene trap insertion in the last intron of the sestrin 2 gene (Ensembl Id: ENSMUSG00000028893) and position of the allele specific primers (arrows). The resulting fusion protein lacks 27 aa of sestrin 2.

FIG. 4: Whole mount E7.5 embryo stained with X-Gal.

FIG. 5: Analysis of sestrin 2 gene expression in WO77E04 mutant mice. A. qRT-PCR of total RNA extracted from lungs of wild type (WT), heterozygous (+/−) and homozygous (−/−) mice. B. Western blot analysis of sestrin 2 protein expression in isolated mouse colon (lanes 1, 2) and lung (lanes 3, 4) fibroblasts using polyclonal anti-mouse sestrin 2 (PTG Inc., Chicago, Ill.) and anti-tubulin antibodies. Minor bands reacting with polyclonal anti-sestrin 2 antibody are most likely non-specific.

FIG. 6: Tracheobronchial silicone casts of 5.5 months old wild type, single ltbp4 and double ltbp4/sesn2 mutant mice.

FIG. 7: Pulmonary emphysema of age matched single ltbp4 double ltbp4/sesn2 mutant mice. HE stain at ×40 magnification.

FIG. 8: Elastin and collagen content in single ltbp4 and double ltbp4/sesn2 mutant lungs. A. Elastin (black) stained with Weigert's resorcin-fuchsin at ×200 magnification. B. collagen (blue) stained with Masson's trichrome stain at ×200 magnification.

FIG. 9: Tracheobronchial silicone cast of an ltbp4^{-/-} lung heterozygous for sesn2 null alleles.

FIG. 10: Collagen deposition in ltbp4 mutant lungs heterozygous (sesn^{+/-}) and homozygous (sesn^{-/-}) for the sesn2 null allele. Collagen (blue) stained with Masson's trichrome stain at ×200 magnification.

FIG. 11: Colorectal adenoma in age matched single (ltbp4^{-/-}) and double (ltbp4^{-/-} sesn2^{-/-} ) mutant mice.

FIG. 12: P-smad2 levels in the colon of age matched single (ltbp4^{-/-}) and double (ltbp4^{-/-}sesn2^{-/-}) mutant mice. Positive cells are reddish-brown.

FIG. 13: P-smad2 levels in sesn2 mutant lungs. Lung section were stained with polyclonal anti-P-smad2 antibody. Positive cells are reddish-brown.

FIG. 14: Increased connective tissue deposition in mutant sesn2 lungs. HE stain at ×200 (top) and ×400 (bottom) magnifications.

FIG. 15: ROS levels in MLFs. A. Basal and H₂O₂-induced (200 μM, 3 hours) ROS amounts measured by FACS (530 nm) after DCF treatment. FL1-H, fluorescence intensity. B. Basal and H₂O₂ induced ROS amounts accumulating in MLFs expressed as mean intensity of cell fluorescence±SD of the two independent experiments shown in A.

### DETAILED DESCRIPTION OF THE INVENTION

The particular terms and abbreviations utilized to define the present invention are further defined in the following.

The terms “does not produce functional sestrins” and “does not produce functional ltbp4” denote the lack of effective sestrin and ltbp4 expression, respectively. A “lack of effective sestrin and ltbp4 expression” also includes the expression of non-functional (i.e. truncated) sestrins and ltbp4 proteins which are not exerting the function of the native protein.

The term “produces suboptimal levels of sestrins” and “produces suboptimal levels of ltbp4” encompasses the translation level of sestrin and ltbp4 proteins, which are insufficient to exert their function. Preferably, the level is reduced at least by 50%, more preferably by 70% and most preferably by 100%.

The term “homozygous disruption” relates to an identical mutation in both alleles of a gene.

The term “heterozygous disruption” relates to a mutation in only one allele of gene. The term “mutation” refers to a change of one or more nucleotide pairs of a DNA molecule.

The term “insertion” is directed to a mutation identified by the presence of one or more additional base pairs in the DNA. The term “deletion” relates to a mutation generated by removal of a sequence of DNA (one or more base pairs), the regions on either side being joined together. The term “substitution mutation” is directed to a nucleotide exchange. The substitution mutation can result in an amino acid change or can introduce a premature translation stop codon. Furthermore, a substitution mutation can affect splicing or expression of the gene when occurring at sites necessary for splicing or gene regulation.

The term “gene targeting” relates to a type of homologous recombination that occurs when a fragment of genomic DNA is introduced into a cell and that fragment recombines with homologous sequences in the genome. The term “gene trap integration” is directed to insertion of a vector, which comprises a reporter gene and which is activated upon insertion into an active transcription unit of the genome.

The term “mutagenesis” denotes a chemical or physical treatment that changes nucleotides in the genome of an organism. An example of a chemical mutagenesis is N-ethyl-N-nitrosurea (ENU) mutagenesis.

The term “exon” encompasses a segment of a gene, which is decoded to give a mRNA product. Individual exons may contain protein coding DNA and/or noncoding DNA. The term “intron” denotes non-coding DNA, which separates neighboring exons in a gene. During gene expression, introns like exons are transcribed into RNA but the transcribed intron sequences are subsequently removed by RNA splicing and are not present in mRNA. The term “regulatory region” relates to the nucleotide sequence, which comprises regions that are necessary for the regulation of gene transcription. These regions comprise, for example, promoters and enhancers and they can be located in 5′ untranslated regions, exons, introns and 3′UTRs. The term “splice site” encompasses the nucleotides at the beginning and the end of the intron that are required for the joining of two exons by removing the intercalated intron during primary transcript processing to functional mRNA.

The term “pulmonary emphysema” denotes a symptom of chronic obstructive lung disease (COPD) characterized by a size increase beyond normal of air spaces distal to the terminal bronchioles and inflammatory infiltrates.

The term “cardiomyopathy” designates a primary non-inflammatory disease of the heart muscle, which is the result of pulmonary hypertension that complicates COPD. The term “cancer” refers to an uncontrolled proliferation of epithelial cells lining the colonic crypts.

“ROS metabolism” refers to the production and neutralization (reduction) of intracellular reactive oxygen species such as hydrogen peroxide and oxygen anions.

The term “profibrotic changes” refers to an increased tissue deposition of collagen associated with a multiplication of fibroblasts.

The term “selecting an agent for treating a symptom” encompasses choosing a composition for management of the condition.

The term “application of one or more agents” relates to administering single compounds or compound combinations orally, by inhalation, parenterally, e.g. intravenously, subcutaneously, intraperitoneally or intramuscularly, or topically, e.g. ophtalmically, vaginally, rectally or intranasally.

The invention is hereinafter described in more detail by referring to the accompanying Figures and Examples.

In a preferred embodiment of aspect (1) of the invention, the genome of the animal comprises a homozygous disruption of the sesn2 and ltbp4 genes. Preferably this homozygous disruption has been generated by a mutation and this mutation can be an insertion, deletion or a substitution mutation. Furthermore, preferably said mutation is generated by gene targeting, gene trapping or chemical mutagenesis and it has occurred in an exon, intron, regulatory region or splice site of the sestrin 2 gene, preferably into the last intron (i.w. the 9^{th} intron) of the sestrin 2 gene, and in an exon, intron, regulatory region or splice site of the ltbp4 gene, preferably in the 5^{th} intron of ltbp4. It is also preferred that the mutation sites rise to an expression of a detectable reporter gene such as fluorescent proteins (such as GFP and its derivatives), enzymes (such as LacZ) or selection markers (such as βgeo), or the like. Particularly preferred is that
(i) the sestrin 2 gene is disrupted in the 9^{th} intron by inserting a gene trap vector, preferably pT1βgeo (SEQ ID NO:10); and/or
(ii) the ltbp4 gene is disrupted in the 5^{th} intron by inserting a gene trap vector, preferably U3Cre (SEQ ID NO:16).

In a further preferred embodiment said animal comprises a heterozygous disruption of the sens2 gene and a homozygous disruption of ltbp4 gene or homozygous disruption of the sestrin 2 gene and a heterozygous disruption of the ltbp4 gene.

In another aspect of the invention the animal model exhibits pulmonary emphysema and/or cardiomyopathy and/or colorectal cancer. Furthermore, the non-human animal is preferably a non-human mammal, more preferably a rodent including mouse and rat.

In a further preferred embodiment the animal model exhibits defects in ROS metabolism and/or profibrotic changes in one or more major organs, preferably in the lung or colon.

In a further preferred embodiment the animal model develops symptoms that are less severe than those exhibited by the animal model disclosed in WO 03/015505 A3.

Embodiment (3) of the invention relates to a cell or tissue culture isolated from a non-human animal model (1) or (2). Preferably, the cell is derived from the lung or colon.

In a preferred aspect of this embodiment, the cell or tissue culture is isolated from a non-human animal model whose genome comprises a homozygous disruption of one or more sestrin genes such that said genes do not produce functional sestrins, preferably sestrin 2 and a homozygous disruption of the ltbp4 gene such that said gene does not produce functional ltbp4.

In another preferred aspect of this embodiment, the cell or tissue culture is isolated from a non-human animal model whose genome comprises a heterozygous disruption of one or more sestrin genes such that said genes do produce only 50% or less of functional sestrins, preferably sestrin 2 and a homozygous disruption of the ltbp4 gene such that said gene does not produce functional ltbp4.

In another preferred aspect of this embodiment, the cell or tissue culture is isolated from a non-human animal model whose genome comprises a homozygous disruption of one or more sestrin genes, preferably sestrin 2 and a heterozygous disruption of ltbp4 such that said gene produces only 50% or less of functional ltbp4. Particularly preferred is that the cell or tissue culture is derived from lung or colon.

Embodiment (4) of the invention relates to a method for preparing the non-human animal model of embodiments (1) and (2), which comprises disrupting the sestrin and/or ltbp4 gene in a germ cell of a starting non-human animal. In a preferred aspect of this embodiment, the non-human animal and the starting non-human animal is a non-human mammal and the germ cell is an ES cell. The method may further comprise introducing the resulting ES cells into blastocysts, injecting the obtained blastocysts into respective non-human foster mothers and intercrossing the resulting chimeras.

Embodiment (5) of the invention is directed to a method for selecting an agent or agents for treating a symptom occurring in the animal model of the invention comprising: (i) applying one or more agents to be tested to the animal model of the invention; and (ii) determining whether one or more symptoms occurring in the animal model of the present invention have changed as a result of the application of said agent or agents. In a preferred embodiment the symptom is selected from a group consisting of pulmonary emphysema, cardiomyopathy and cancer. In a further aspect of the invention the agent, which is suitable for treating a symptom occurring in the animal model of the invention is a pharmaceutical. Furthermore, the present invention relates to the use of the agent, which is suitable for treating a symptom occurring in the animal model of the invention for the preparation of a pharmaceutical composition for the treatment of pulmonary emphysema. Moreover, the present invention is directed to a method of treatment of cancer and pulmonary emphysema using the agent, which is suitable for treating a symptom occurring in the animal model of the present invention.

Embodiment (6) of the invention is directed to a method selecting an agent that interferes with ROS production and TGF-β signaling comprising:
(i) applying one or more agents to be tested to the to the cell or tissue culture of (3) above,
(ii) determining whether cellular ROS levels and TGF-β signaling have changed as a result of the application of said agents or agents.

It is particularly preferred in said method that the cell or tissue culture is from lung or colon.

Embodiment (7) of the invention is a method to analyze whether pulmonary emphysema and/or cancer is caused by differential ltbp4 and sestrin gene or protein expression or expression level or by a defect in the ltbp4 and sestrin genes comprising the (i) characterization of the ltbp4 and sestrin gene or protein expression or expression level or of the ltbp4 and sestrin gene allele status of an individual having pulmonary emphysema and/or cancer, and the (ii) characterization of the ltbp4 and sestrin gene or protein expression or expression level or ltbp4 and sestrin gene allele status of a control individual. A difference in the ltbp4 and sestrin gene or protein expression or expression level or in the ltbp4 and sestrin gene allele status indicates that a defect in the ltbp4 and sestrin genes is involved in the pathogenesis of pulmonary emphysema and/or cancer.

Embodiment (8) of the invention relates to a method for diagnosing pulmonary emphysema or cancer comprising the (i) characterization the ltbp4 and sestrin gene or protein expression or expression level or the ltbp4 and sestrin gene allele status of an individual and (ii) characterization of the ltbp4 gene or protein expression or expression level or ltbp4 and sestrin gene allele status of a control individual. A difference in the ltbp4 and sestrin gene or protein expression or expression level or in the ltbp4 and sestrin gene allele status would indicate the presence of pulmonary emphysema and/or cancer in said individual. Among the determined expression levels it is the ltbp4 expression level that is an indicator of the severity of the pulmonary emphysema or of the cancer, while the sestrin expression level is a marker for the disease progression such that low expression levels are beneficial to the disease progression.

“Individual” used in connection with the embodiments (7) and (8) relates to an individual with suspected abnormal ltbp4 and sestrin gene allele status, i.e. a patient. “Control individual” refers to a healthy individual having normal ltbp4 and sestrin allele status.

In a preferred embodiment of the methods to analyze whether pulmonary emphysema and/or cancer are linked to ltbp4 and sestrin and for diagnosing pulmonary emphysema and cancer, the ltbp4 and sestrin gene allele status and the ltbp4 and sestrin expression or expression level are detected by genomic PCR, RT-PCR, Northern analysis, microarray (DNA chip) analysis or antibodies directed to the ltbp4 and sestrin proteins.

The methods of embodiments (5) to (8) are suitable to be performed in vivo and in vitro.

The animal model of the present invention can be used to dissect the molecular mechanisms controlling the sestrin/TGF-β pathway and for the identification and cloning of modifier genes able to modify, aggravate, reduce or inhibit the phenotype associated with pulmonary emphysema and cancer or other conditions occurring in the animal model of the invention. Moreover, the animal model can be used for identification of early diagnostic markers for cancer and/or pulmonary emphysema or other conditions occurring in the animal model of the invention. In addition, the animal model of the present invention can be used for the monitoring of the activity of agents useful in the prevention or treatment of cancer and/or pulmonary emphysema or other conditions occurring in the animal model of the invention and as a test model system for agents suspected of promoting or aggravating cancer and/or pulmonary emphysema or other conditions occurring in the animal model of the invention.

The invention furthermore explained in the following examples which are, however, not to be construed as a limitation of the invention.

### EXAMPLES

### Methods

Cell cultures: [129/SvPas] strain derived ES cells were grown on irradiated (32 Gy) MEF feeder layers in DMEM supplemented with 15% (v/v) preselected and heat inactivated fetal calf serum (FCS) (Linaris, Bettingen, Germany), 100 mM nonessential amino acids (Gibco), 0.1 mM mercaptoethanol (Sigma), 1000 U/ml of leukemia inhibitory factor (LIF) (Esgro®; Gibco/BRL), as described (De-Zolt S., et al. Nucleic Acids Res. 34:e25, 2006). Lung and colon fibroblast cultures from adult wild-type and sens−/− mice were established according to standard protocols as previously described (Koli K., et al. J Cell Biol. 167:123-33, 2004) and grown in Dulbeccos's modified Eagle's medium (DMEM) supplemented with 10% fetal calf serum (Life Technologies), 100 IU/ml penicillin and 50 μg/ml streptomycin. During the first two weeks in culture spontaneous immortalization occurred generating cell lines.

5′ RACE and sequencing: cDNAs from gene trap expressing ES cell lines were prepared from the polyadenylated RNA using a RoboAmp robotic device (MWG Biotech, Ebersberg, Germany) with a processing capacity of 96 samples/day. Samples of 2×10⁵ cells were lysed in 1 ml of lysis buffer containing 100 mM Tris/HCl pH 8.0, 500 mM LiCl, 10 mM EDTA, 1% LiDS and 5 mM DTT. Polyadenylated RNA was captured from the lysates by biotin-labeled oligo-d(T)-primers according to the manufacturers instructions (Roche Diagnostics Corp., Indianapolis, Ind., USA) and placed on streptavidin-coated 96-well plates (AB Gene, Surrey, UK). After washing, solid-phase cDNA synthesis was performed in-situ using random hexamers and Super-Script II RT (Invitrogen, Karlsruhe, Germany). To remove excess primers the cDNAs were filtered through Multiscreen PCR plates (Millipore Corp. Bedford, Mass., USA). The 5′ ends of the purified cDNAs were tailed with dCTPs using terminal transferase -TdT- (Invitrogen, Karlsruhe, Germany) following the manufacturer's instructions. For PCR amplification of the cellular sequences appended to the gene trap transcripts the following vector-specific primers were used: 5′-CTA CTA CTA CTA GGC CAC GCG TCG ACT AGT ACG GGI IGG GII GGG IIG-3′ (SEQ ID NO:4) and 5′-GCC AGG GTT TTC CCA GTC ACG A-3′ (SEQ ID NO:5); and for nested PCR: 5′-CTA CTA CTA CTA GGC CAC GCG TCG ACT AGT AC-3′ (SEQ ID NO:6) and 5′-TGT AAA ACG ACG GCC AGT GTG AAG GCT GTG CGA GGC CG-3′ (SEQ ID NO:7). Amplification products were directly sequenced using AB377 or ABI3700 sequencing machines (Applied Biosystems ABI, Foster City, USA).

ES cell injections, breeding and genotyping: WO77E06 (TBV-2; 129SvPas) ES cell derived chimeras were generated by injecting C57Bl/6 blastocysts. The resulting male chimeras were bred to C57Bl/6 females, and F1 agouti offspring containing the disrupted transgene were intercrossed to obtain homozygous F2 mice. Genotyping was performed on mouse tail DNA by genomic PCR using primers against the sequences flanking the gene trap insertion previously identified in ES cells by inverse PCR and sequencing.

Histology, histochemistry and immunohistochemistry: Paraffin sections of mouse tissues were prepared and stained using standard histology procedures. To visualize elastin and collagen fibers, microscopic slides were stained with Weigert's resorcin-fuchsin or Masson's trichrome stain as previously described (Sterner-Kock A., et al. Genes Dev. 16:2264-2273, 2002). Immunostainings using rabbit polyclonal anti- P-Smad2 antibodies were performed as described in Sterner-Kock et al (Ster ner-Kock A., et al. Genes Dev. 16:2264-2273, 2002)

Plastinations: Tracheas of lungs prepared from wild-type and mutant mice were injected at room temperature with 1-2 ml of E RTV silicone (Dow Corning, Midland, Mich., USA) using a 2 ml syringe. Curing Agent was added at a 1:10 ratio to the silicone polymer immediately prior to injection. Injection was stopped when the silicone was visible under the surface of both lungs. Following injection, the silicone was allowed to harden for 24-48 hours after which the specimens were placed in 10% potassium hydroxide solution for 5-7 days. and then left in boiling water for 8-12 hours to detach the tissue from the polymer. Following maceration in boiling water, the specimens were placed in 5% hydrogen peroxide for about 2 hours to complete the removal of residual tissues. Casts were then rinsed in running water overnight.

RNA isolation, RT-PCR and protein analysis: Total cellular RNA was isolated using RNeasy Mini kit (Qiagen) according to manufacturer's instructions. RNA concentrations and purities were determined spectrophotometrically (Ultrospec 3000, Amersham) as well as by agarose gel electrophoresis followed by ethidium bromide staining. RT-PCRs were performed according to standard protocols by using 75 ng of reverse-transcribed total RNA in a total volume of 50 μl. Real-time RT-PCR analysis of sestrin 2 gene expression in ES cells was performed using SYBR green chemistry (ABgene, Epsom, UK) and an iCycler (Biorad) machine. cDNA was synthesized from total RNA using random priming and Superscript II (Invitrogen) reverse transcriptase. PCR reactions were run as triplicates in 25-μl volumes on 96-well plates, with each reaction containing cDNA derived from 15 ng of total RNA, 1× ABsolute SYBR fluorescein mix (ABGene) and 5 pmol of each of the following primers: 5′-CCTGGAACGGAACCTCAAAATC-3′ (SEQ ID NO:8) and 5′-GGGCTTCAAGGAGCAGCAAG-3′(SEQ ID NO:9). Amplification reactions were allowed to proceed for 35 cycles at 94° C. for 15 s, 61° C. for 30 s, and 72° C. for 30 s. For Western blotting lysates of lung and colon fibroblasts were resolved by SDS-PAGE, transferred to nitrocellulose membranes and reacted with rabbit anti-sestrin 2 polyclonal antibody (PTG Inc., Chicago, Ill.).

Determination of intracellular peroxide levels: 3×10⁵ lung or colon fibroblasts from wild type and sesn2^{-/-} mice plated into 6 cm Petri dishes a day before were trypsinized, washed in serum free DMEM and resuspended in 5 ml of serum free DMEM. To some cultures H₂O₂ was added to a final concentration of 200 μM. After incubating for 3 hours dichlorodihydrofluorescein (DCF) was added to the cells at a final concentration of 30 μM and incubated for another hour. After that cell were washed in PBS, trypsinized and resuspended in 300 μl PBS which were subjected to FACS analysis.

### Example 1

### A pT1βgeo Gene Trap Insertion in the Sestrin 2 Gene Induces a Null Mutation in Transgenic Mice

The WO77E06 gene trap ES cell line was obtained by electroporating the pT1βgeo gene trap vector shown in SEQ ID NO:10 into TBV-2 (129SvPas) ES cells as previously described (Floss, T. & Wurst, W. Methods Mol Biol 185, 347-79, 2002). Gene trap expressing ES-cell clones were selected in 200 μg/ml G418 (Gibco/BRL), manually picked, expanded, and stored frozen in liquid nitrogen. The gene trap insertion in the 9^{th} intron of the sestrin 2 (sesn2) gene was identified among the recovered clones by 5′ RACE as described in the Methods section and by sequence database analysis (Genbank at http://www.ncbi.nlm.nih.gov) using the BlastN algorithm. The gene trap insertion in the WO77E06 cell line was verified by genomic PCR (see Methods) using the following primers complementary to gene trap- and adjacent upstream intron sequences: 5′-CAGCCTTGAGCCTCTGGAGC-3′ (SEQ ID NO:11) and 5′-CTACCCTGAGAAGACGACCCG-3′ (SEQ ID NO:12). The verified WO77E06 ES cells were then converted into mice by blastocyst injection. F1 mice carrying the gene trap allele were intercrossed and the F2 offspring was mated to ltbp4^{-/-} mice (WO 03/015505 A3) to obtain double knock-out mice.

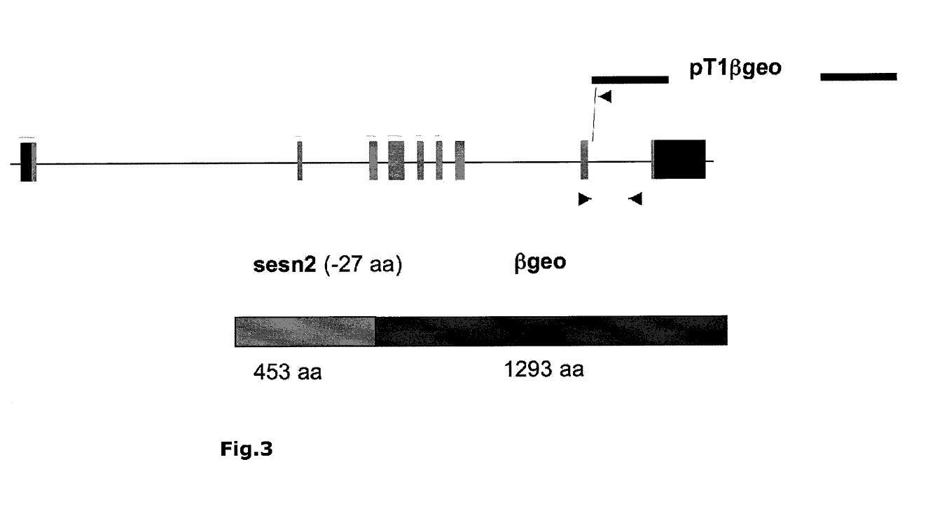

FIG. 3 shows the genotyping strategy for the WO77E06 (sesn2) mice. Tail DNA was PCR amplified in parallel reactions using a forward primer in the 9^{th} exon (5′-CTACCCTGAGAAGACGACCCG-3′; SEQ ID NO:13) and two reverse primers; one in the 9^{th} intron for detecting the wild type allele (5′-GGACAAATCAAGGTTACACAGAAAAAAGTC-3′; SEQ ID NO:14) and the other in the gene trap's splice acceptor site for detecting the gene trap allele (5′-CAGCCTTGAGCCTCTGGAGC-3′; SEQ ID NO:15). Amplification reactions were allowed to proceed for 30 cycles at 94° C. for 15 s, 61° C. for 30 s, and 72° C. for 30 s.

Intercrossing F1 heterozygous offspring yielded homozygous offspring at a frequency consistent with a Mendelian inheritance pattern of the disrupted transgene, indicating that sesn2 is not required for development. Mutant mice developed normally after birth and were grossly indistinguishable from their wild type and heterozygous littermates.

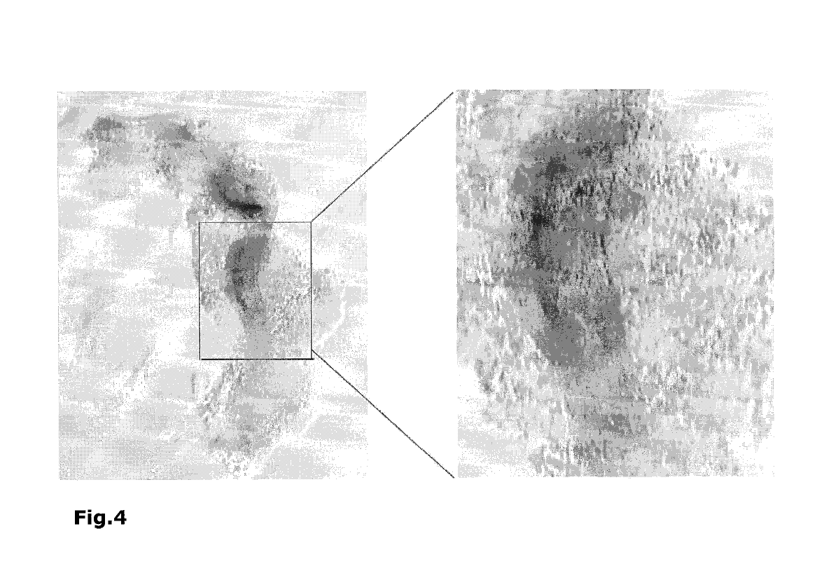

The gene trap insertion in the 9^{th} intron of the sesn2 gene induces a fusion transcript in which the exons upstream of the insertion are spliced in frame to βgeo. Because transcription is terminated at the gene trap's polyadenylation site, the processed fusion transcript encodes a truncated version of sesn2 and the βgeo reporter (FIG. 3). FIG. 4 shows faint expression of this protein in an early heterozygous embryo stained with X-Gal.

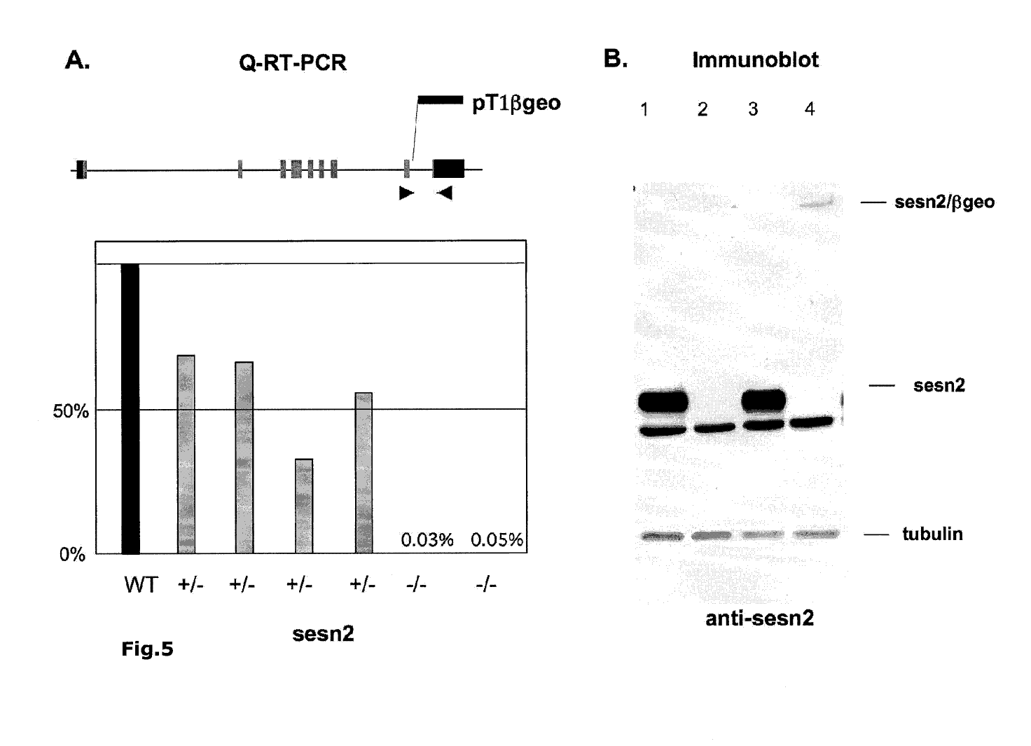

Wild type sesn2 transcripts were completely lost from lungs of homozygous WO77E06 mice whereas heterozygous lungs expressed about 50% of wild type levels (FIG. 5A). Moreover, in isolated lung (MLF) and colon (CLF) fibroblasts, which both express high levels of sesn2 (FIG. 5B), no protein was detected in cells from homozygous WO77E06 mice, suggesting that the gene trap insertion had induced a null mutation (FIG. 5B). Consistent with the faint β-galactosidase expression in the early embryos (FIG. 4), mutant MLFs expressed only trace amounts of the sesn2/βgeo fusion protein, implying that the relatively large protein is unstable (FIG. 5B). Given its low expression, this protein is unlikely to have a dominant negative effect even if the truncated protein retained some residual function.

### Example 2

### Sesn2 Null Alleles Improve the Pulmonary Emphysema in Ltbp4^{-/-} Mice

129/Sv (D3) ES cells with a U3Cre gene trap vector (SEQ ID NO:16) insertion in the 5^{th} intron of the ltbp4 gene were injected C57BL/6 blastocysts as previously described (WO 03/015505, Thorey, I. S. et al. Mol Cell Biol 18, 3081-3088, 1998). The resulting male chimeras were bred to C57BL/6 females and agouti offspring were tested for transgene transmission by tail blotting. Mouse tail DNA was cleaved with BglII which does not cut provirus. The DNA was fractionated on 1% agarose gels, blotted onto Hybond N nylon filters (Amersham/Pharmacia, Piscataway, N.J.) and hybridized to a ³²P labeled provirus flanking sequence probe. Animals heterozygous for the gene trap insertion were backcrossed to C57BL/6 mice for at least six generations before analyzing the phenotypes in heterozygous and homozygous offspring. To obtain double mutant strains, heterozygous ltbp4^{+/-} mice were crossed to homozygous sesn2^{-/-} mice and the offspring was genotyped by tail DNA PCR as described in Example 1 using the following allele specific primers: ltbp4 wild type allele=5′-CCAATCTTGCTTCTTTGCTGAGC-3′ (SEQ ID NO:17) and 5′-GGCTCATGCTTGAATGTTTCAG-3′ (SEQ ID NO:18); ltbp4 gene trap (mutant allele)=5′-CCAATCTTGCTTCTTTGCTGAGC-3′ (SEQ ID NO:19) and 5′-ATCATGCAAGCTGGTGGCTG-3′ (SEQ ID NO:20); sesn2 wild type allele=5′-CTACCCTGAGAAGACGACCCG-3′ (SEQ ID NO:21) and 5′-GGACAAATCAAGGTTACACAGAAAAAAGTC-3′ (SEQ ID NO:22); sesn2 gene trap (mutant allele)=5′-CTACCCTGAGAAGACGACCCG-3′ (SEQ ID NO:23) and 5′-CAGCCTTGAGCCTCTGGAGC-3′ (SEQ ID NO:24).

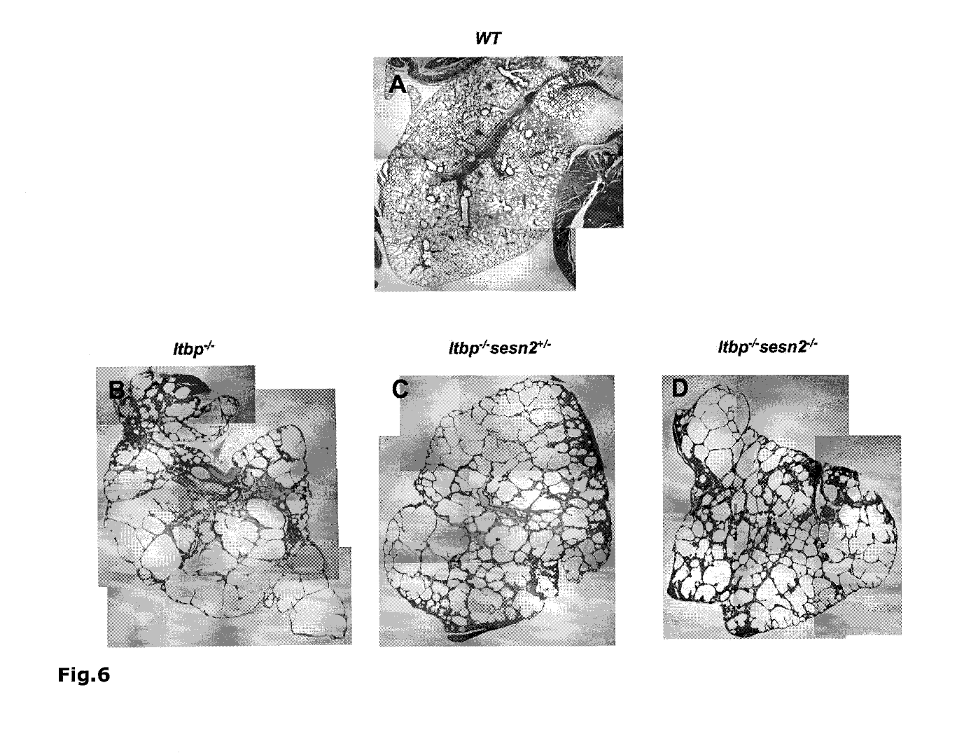

FIG. 6B shows a typical emphysematous lung of an adult Itbp4^{-/-} mouse (WO 03/015505). It displays massively dilated alveoli surrounded by thin, dysplastic and frequently disrupted septal walls. The lobular connective tissue is significantly reduced, and lungs display multifocal atelectatic areas. Consistent with a partial recovery alveolar spaces in double mutant Itbp4^{-/-}sesn^{-/-} littermates were more numerous, reduced in size and separated by thicker walls (FIG. 6D).

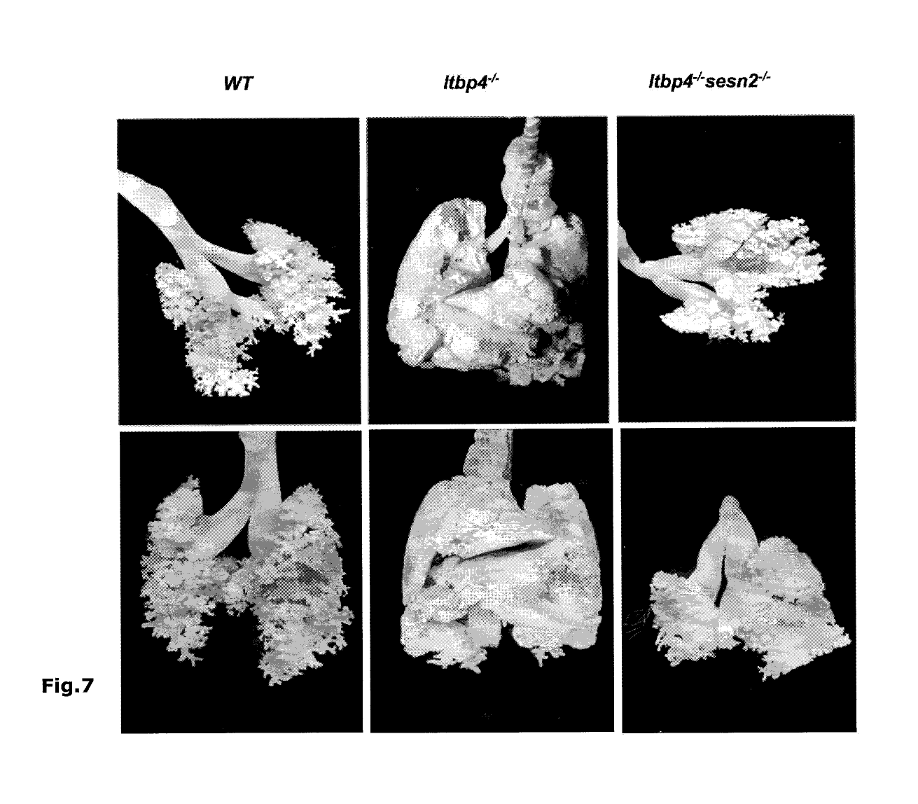

To detect the extent of damage to the bronchioles, alveolar ducts and alveoli, we visualized the tracheobronchial trees of these animals by plastination. Plastination involves the instillation of silicone into the trachea of the isolated lung. Since the alveolar ducts prevent the silicone from entering the alveolar sacs, the technique provides an informative, three dimensional image of the tracheobronchial tree (Perry S. F., et al. Exp Lung Res. 26:27-39, 2000). FIG. 7 (left panels) shows the tracheobronchial tree of two 5.5 months old wild type mice with ramifications down to the terminal bronchioles and alveolar ducts. In mutant ltbp4 littermates, these ramifications were almost completely obscured by enlarged airspaces filled with silicone, suggesting that the terminal bronchioles and alveolar ducts are enlarged and leaky (FIG. 7, middle panels). However, a dramatic improvement in the tracheobronchial architecture was observed in ltbp4^{-/-} mice carrying two sesn2 null alleles (Itbp4^{-/-}sesn2^{-/-}) (FIG. 7, right panels). In contrast to an almost invisible tracheobronchial architecture in ltbp4^{-/-} mice, the double mutant tracheobronchial tree was close to normal again, implying a regeneration of terminal bronchioles and alveolar ducts. These modification were much more dramatic than the parenchymal changes presumably reflecting an uneven regeneration of collagen and elastin fibers, which are the major components of the pulmonary ECM (Suki B., et al. J Appl Physiol. 98:1892-9, 2005). Although both form dense fiber networks throughout the lung, the elastin fibers are distributed evenly whereas the collagen fibers tend to condense around terminal bronchioles and alveolar ducts (Toshima M., et al. Arch Histol Cytol. 67:31-40, 2004). Based on this, we speculated that the preferential recovery of the tracheobronchial tree in the ltbp4^{-/-}sesn2^{-/-} mice could be the result of an excess collagen deposition into the pulmonary ECM.

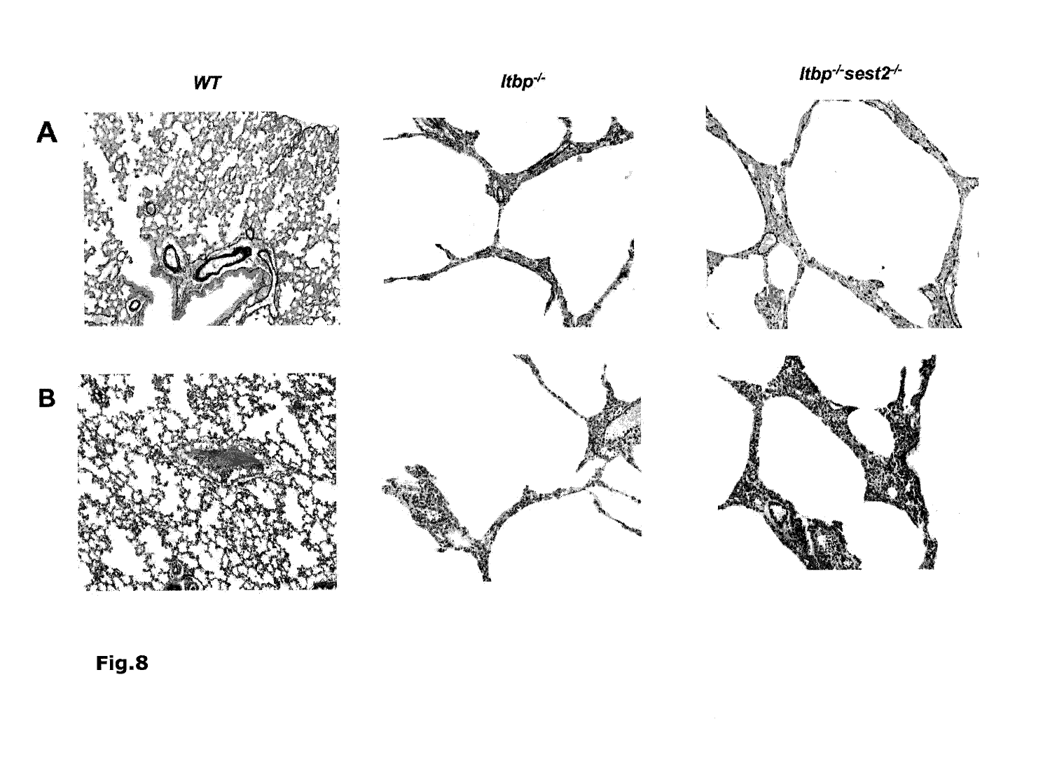

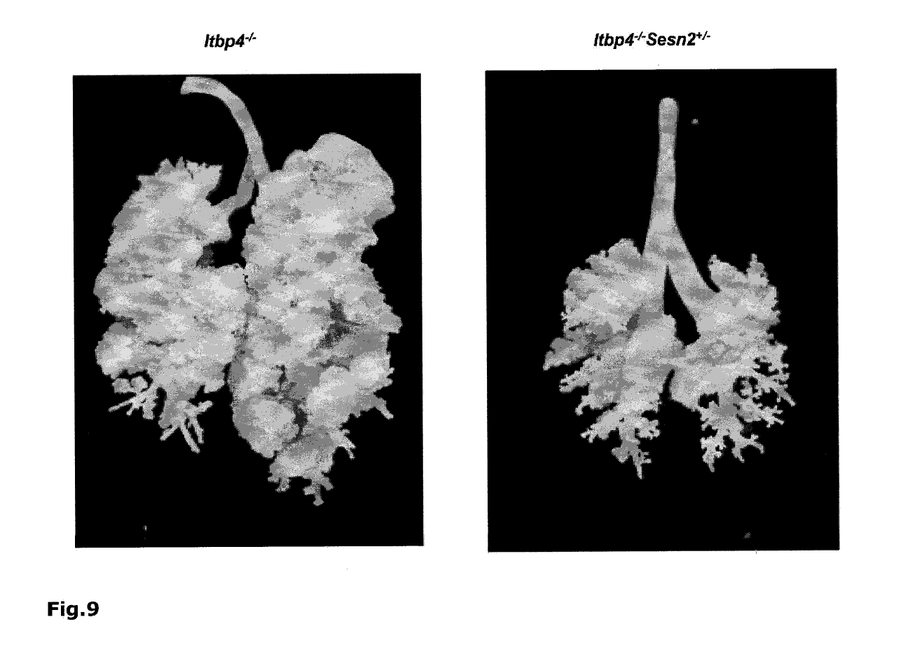

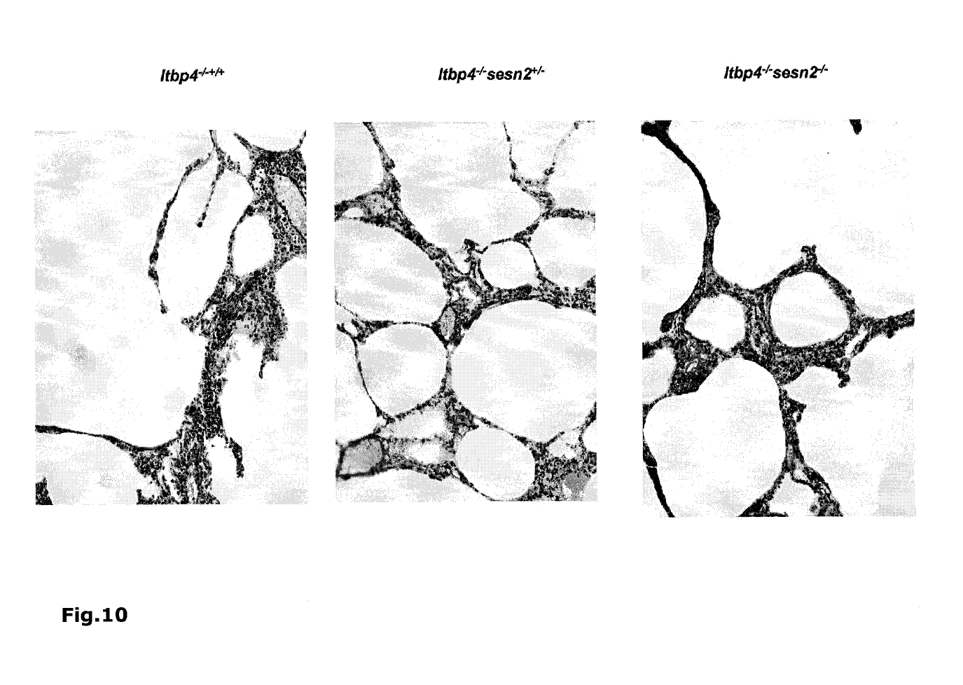

To test this, we visualized elastin and collagen in lung tissue sections using specific histochemical stains. FIG. 8A shows the elastin network of ltbp4^{-/-} and ltbp4^{-/-}sesn2^{-/-} lungs appearing fragmented, patchy and condensed in both. In contrast, collagen deposition was dramatically increased in the double mutant lungs (FIG. 8B). As TGF-β is one of the most potent collagen inducers, the excessive deposition of collagen suggested a reactivation of TGF-β signaling in the ltbp4^{-/-}sesn2^{-/-} lungs. Since tracheobronchial recovery and increased deposition of collagen was also noticed in ltbp4^{-/-} mice heterozygous for sesn2 (ltbp4^{-/-}sesn2^{+/-}) (FIGS. 9, 10), the sesn2 mutation could be haploinsufficient.

### Example 3

### Sesn2 Null Alleles Improve the Rectal Prolapse and Colorectal Adenomas in Ltbp4^{-/-} Mice

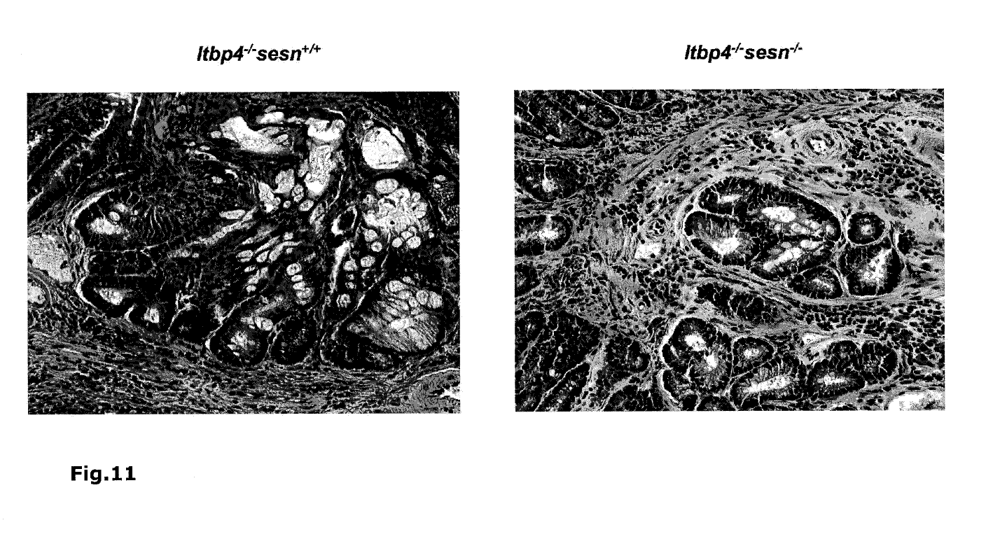

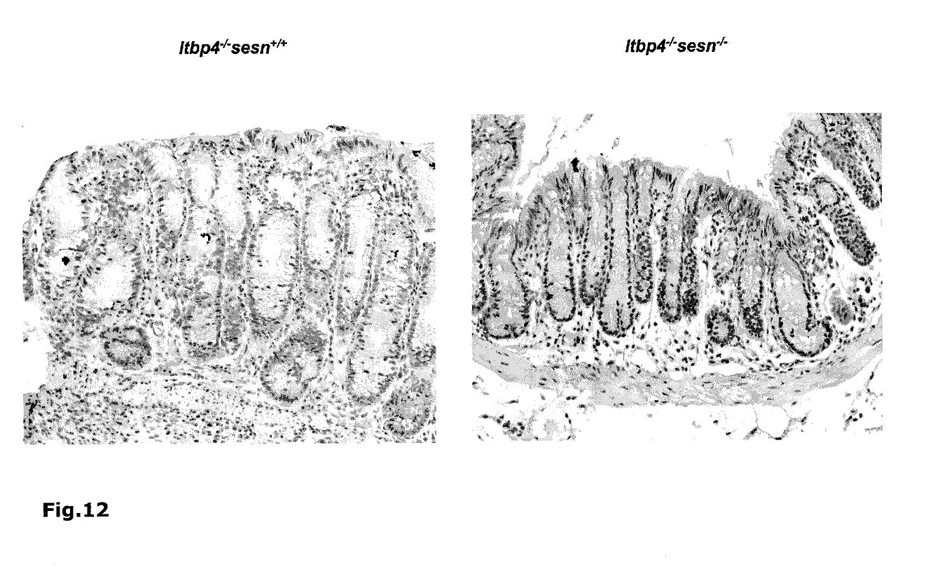

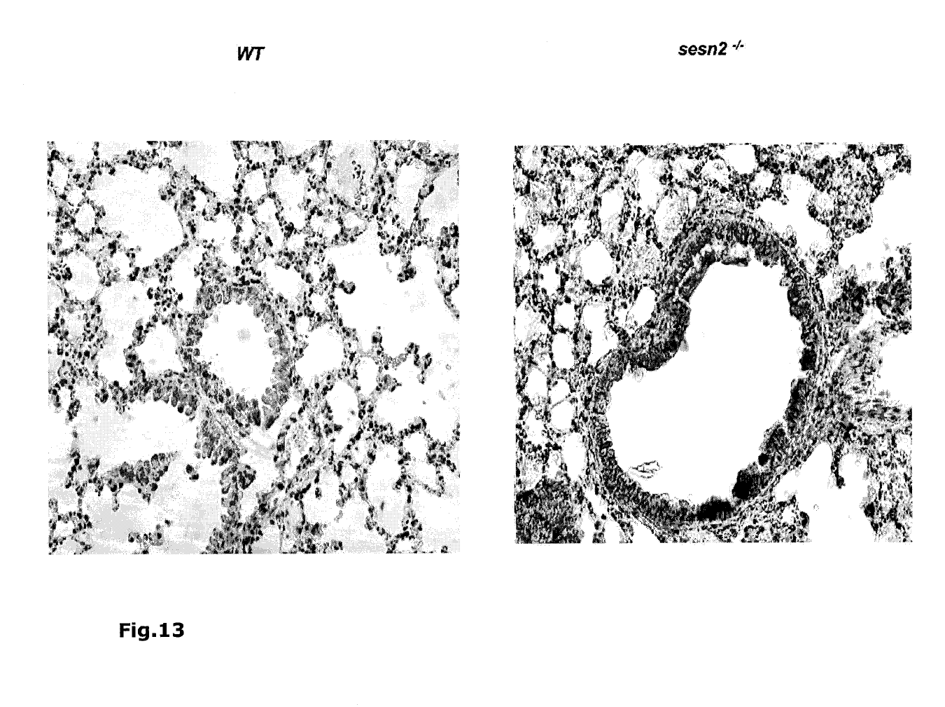

FIG. 13 shows a typical colorectal an adenoma of 3 months old ltbp4^{-/-} (WO 03/015505 A3) mouse. Microscopically, the region exhibited abberant crypt foci containing regenerating epithelial cells and an increased number of goblet cells. Although double mutant ltbp4^{-/-}sesn^{-/-} littermates also exhibited adenomas, these were significantly smaller in size and contained fewer goblet cells (FIG. 11), suggesting a partial phenotypic rescue. Since the ltbp4−/− phenotype is essentially caused by defective TGF-fβ activation (Sterner-Kock A., et al. Genes Dev. 16:2264-2273, 2002), we tested whether the partial rescue was associated with a reactivation of TGF-fβ signaling. Towards this end we determined the levels of phosphorylated smad2 in tissue sections by immunohistochemistry. Consistent with an activation of TGF-fβ signaling in ltbp4^{-/-}sesn2^{-/-} mice, p-smad2 levels were very high in contrast to the single mutant ltbp4^{-/-} mice, which have no detectable P-smad2 in their colon (FIG. 12).

### Example 4

### Sesn2^{-/-} Lungs Exhibit Enhanced TGF-β Signaling

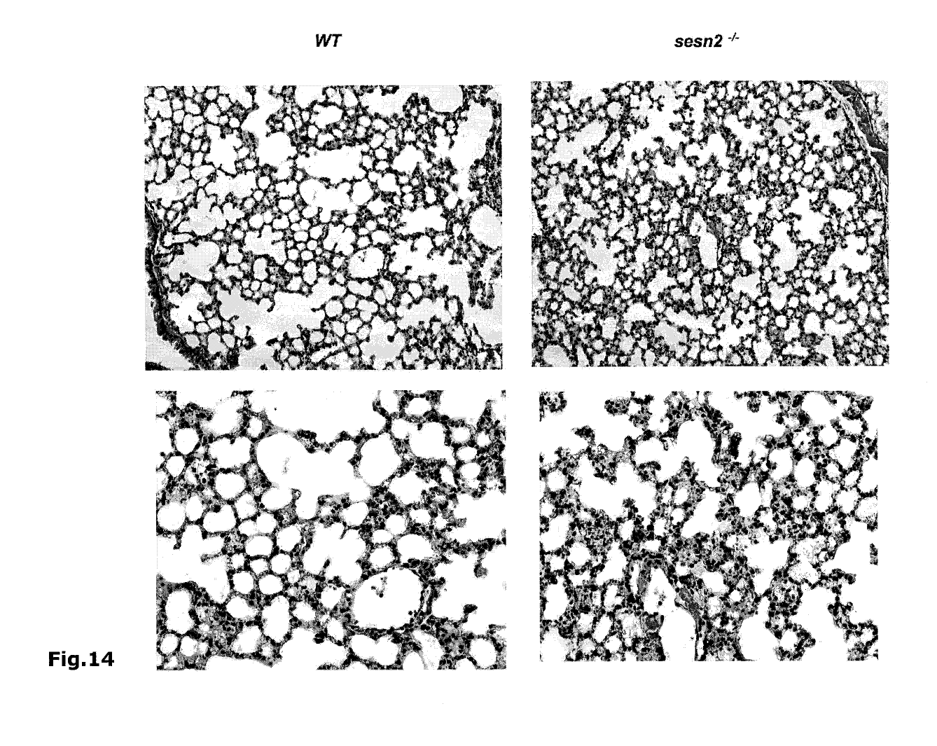

The increased collagen deposition in the lung and the increased P-smad2 levels in the colon of ltbp4^{-/-}sesn2^{-/-} mice suggested the TGF-β pathway might be similarly activated in the sesn2^{-/-} mice despite their grossly normal phenotype. To test this we visualized P-smad2 in lung tissue sections as described above. In a preliminary experiment, we found that P-smad2 levels in sesn^{-/-} lungs significantly exceeded the wild type levels, suggesting that loss of sestrin 2 activates TGF-fβ (FIG. 13). Moreover, although we could not detect increased collagen deposition by Masson's trichrome stain (data not shown), the sesn2^{-/-} lungs exhibited more abundant connective tissue, smaller airspaces and thicker interalveolar walls, which are all consistent with incipient, TGF-fβ induced fibrosis (FIG. 14) Sime P. J., et al. J Clin Invest. 100:768-76, 1997; Lee M. S., et al. Am J. Pathol. 147:42-52, 1995; Sanderson N., et al. Proc Natl Acad Sci USA. 92:2572-6, 1995).

### Example 5

### Increased Peroxide Accumulation in Mouse Lung Fibroblasts (MLFs) Derived from Sesn^{-/-} mice

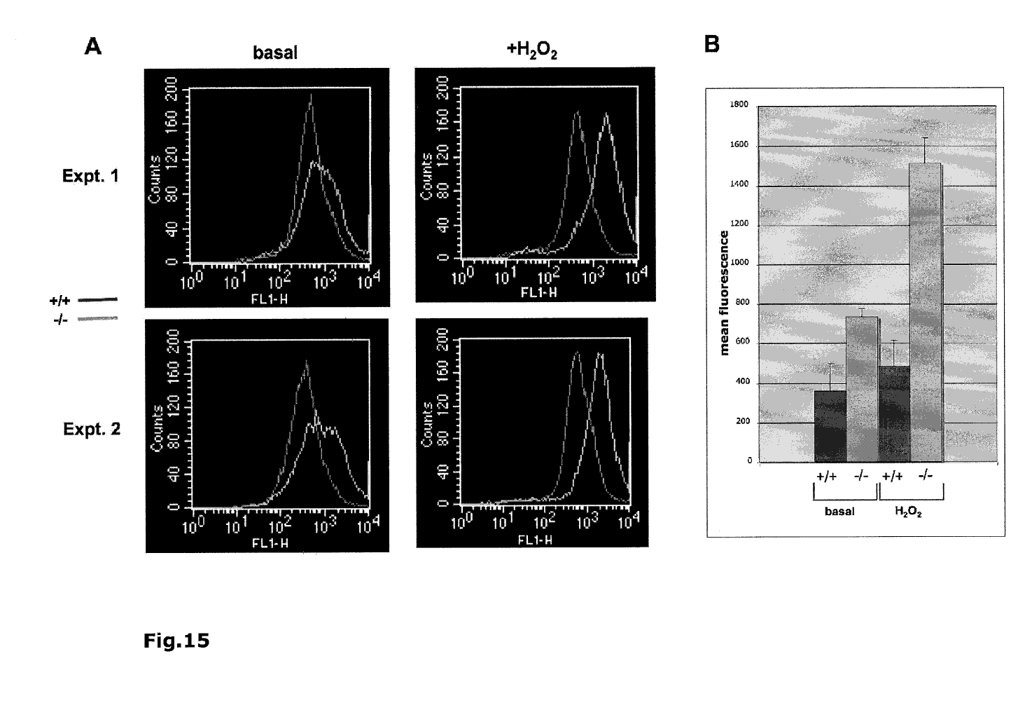

Recently published experiments in have shown that the inhibition of sesn2 expression by shRNA compromises the cell's ability to process ROS leading to ROS accumutation and oxidative stress (Budanov A. V., et al. Science. 304:596-600, 2004). In line with this, we expected antioxidant functions to be similarly compromised in cells derived from sesn2 knock out mice. To test this, we quantified basal and H₂O₂ induced ROS levels in MLFs using the dichlorodihydrofluorescein (DCF) fluorescence method. FIG. 15 shows that sesn^{-/-} MLFs accumulated significantly more ROS than the corresponding wild type cells, regardless of pretreatment with 200 mM H₂O₂. The results are equivalent to those obtained in the previously described shRNA knock down experiments and are consistent with a sesn2 loss of function in the mutant MLFs.

### Sequence Listing, Free Text

### SEQ ID NOs:1-9 Primer

SEQ ID NO:10 Gene trap vector pTβgeo
plasmid Elements:
   En-2 Splice Acceptor 2284-4163
   βGeo 4164-8053
   SV40 pA 8054-8496
   plasmid backbone 8497-2283

### SEQ ID NOs:11-15 Primer

SEQ ID NO:16 Gene trap vector U3Cre
Plasmid elements:
   LTRs 1-1475 and 3046-4520
   Cre 3-1090 and 3074-4133
gag/env: 1475-3045.
   SEQ ID NOs:17-24 Primer

1. A non-human animal model, which does not express one or more functional sestrins or expresses suboptimal levels of one or more sestrins and does not express latent transforming growth factor p binding protein 4 (Itbp4) or expresses suboptimal levels of Itbp4.

2. The animal model of claim 1, wherein
(i) the genome of the animal comprises a homozygous or a heterozygous disruption of a sestrin gene; and/or
(ii) the genome of the animal comprises a homozygous or a heterozygous disruption of the Itbp4 gene.

3. The animal model of claim 2, wherein the disruption being generated by a mutation, wherein said mutation
(i) is an insertion, deletion or substitution mutation; and/or
(ii) is generated by gene targeting, gene trapping or chemical mutagenesis; and/or
(iii) has occurred in an exon, intron, regulatory region or splice site of the sestrin and Itbp4 genes; and/or
(iv) gives rise to the expression of a reporter gene; and/or
(v) has occurred in the 9^{th} ntron of the sestrin 2 gene; and/or
(vi) has occurred in the 5^{th} ntron of the Itpb4 gene.

4. The animal model of claim 2, wherein
(i) the sestrin 2 gene is disrupted in the 9^{th} intron by inserting a gene trap vector; and/or
(ii) the Itbp4 gene is disrupted in the 5^{th} intron by inserting a gene trap vector.

5. The animal model of claim 1, wherein
(i) said non-human animal is a non-human mammal; and/or
(ii) said animal exhibits pulmonary emphysema and cancer.

6. The animal model of claim 1, which does not express one or more functional sestrins.

7. A cell or tissue culture isolated from the animal models of claim 1.

8. The cell or tissue culture of claim 7, wherein the cell or tissue culture is from lung or colon.

9. A method for preparing the non-human animal model of claim 1, which comprises disrupting the sestrin and/or Itbp4 gene in a germ cell of a starting non-human animal.

10. The method of claim 9, wherein the non-human animal is a non-human mammal and the germ cell is an ES cell.

11. A method for selecting an agent for treating a symptom occurring in the non-human animal model of claim 1 comprising:
(i) applying one or more agents to be tested to said animal model,
(ii) determining whether one or more symptoms occurring in said animal model have changed as a result of application of said agent(s).

12. The method of claim 11, wherein the symptom is selected from a group consisting of cancer and pulmonary emphysema.

13. A method for selecting an agent that interferes with ROS production and TGF-β signaling comprising:
(i) applying one or more agents to be tested to the to the cell or tissue culture of claim 7,
(ii) determining whether cellular ROS levels and TGF-β signaling have changed as a result of the application of said agents or agents.

14. The method of claim 13, wherein the cell or tissue culture is from lung or colon.

15. A method to analyze whether cancer and/or pulmonary emphysema is caused by differential Itbp4 and sestrin gene or protein expression or expression level or by a defect in the ltbp4 and sestrin gene comprising:
(i) characterizing the Itbp4 and sestrin gene or protein expression or expression level or Itbp4 and sestrin gene allele status of an individual having cancer or pulmonary emphysema,
(ii) characterizing the ltbp4 and sestrin gene or protein expression or expression level or Itbp4 and sestrin gene allele status of a control individual, a difference in the Itbp4 and sestrin gene or protein expression or expression level or Itbp4 and sestrin gene allele status indicating that cancer and/or pulmonary emphysema and/or cardiomyopathy is linked to differential Itbp4 and sestrin gene or protein expression or expression level or a defect in the Itbp4 and sestrin genes.

16. A method for diagnosing cancer and/or pulmonary emphysema and/or cardiomyopathy comprising:
(i) characterizing the Itbp4 and sestrin gene or protein expression or expression level or Itbp4 and sestrin gene allele status of an individual,
(ii) characterizing the Itbp4 and sestrin gene or protein expression or expression level or ltbp4 and sestrin gene allele status of a control individual, a difference in the LTBP-4 gene or protein expression or expression level or Itbp4 and sestrin gene allele status indicating the presence of cancer and/or pulmonary emphysema and/or cardiomyopathy in said individual.

17. The method of claim 15, wherein
(i) the Itbp4 and sestrin expression or expression level is detected by RTPCR, Northern analysis, microarray analysis or antibodies directed to Itbp4 and sestrin proteins; and/or
(ii) the Itbp4 and sestrin gene expression or expression level is detected by RT-PCR, Northern analysis, microarray analysis or antibodies directed to Itbp4 and sestrin proteins; and/or
(iii) the Itbp4 and sestrin gene allele status is detected by mutation screening.

18. A kit for diagnosing cancer and/or pulmonary emphysema and/or cardiomyopathy comprising means to detect the Itbp4 and sestrin gene or protein expression or expression level or Itbp4 and sestrin gene allele status of an individual and control.

19. The method of claim 16, wherein
(i) the Itbp4 and sestrin expression or expression level is detected by RTPCR, Northern analysis, microarray analysis or antibodies directed to Itbp4 and sestrin proteins; and/or
(ii) the Itbp4 and sestrin gene expression or expression level is detected by RT-PCR, Northern analysis, microarray analysis or antibodies directed to Itbp4 and sestrin proteins; and/or
(iii) the Itbp4 and sestrin gene allele status is detected by mutation screening.


SEQUENCE LISTING (24 sequences)

SEQ ID NO: 1 - protein, 492 aa, Mus musculus
MRLAAASNEAYAASLAVSELLSCHQCGGDRGQDEELGIRIPRPLGHGPSRFIPEKEMLQVGSEDAQMHALFADSFAALGRLDNITLVMVFHPQYLESFLKTQHYLLQMDGPLPLHYRHYIGIMAAARHQCSYLVNLHVSDFLHVGGDPKWLNGLENAPQKLQNLGELNKVLAHRPWLITKEHIEGLLKAEEHSWSLAELVHAVVLLTHYHSLASFTFGCGISPEIHCDGGHTFRPPSVSNYCICDITNGNHSVDEMQVNSAGNASVSDSFFEVEALMEKMRQLQECREEEEASQEEMASRFEMEKRESMFVFSSDDDEVTPARDVSRHFEDTSYGYKDFSRHGMHVPTFRVQDYCWEDHGYSLVNRLYPDVGQLIDEKFHIPYNLTYNTMAMHKDVDTSMLRRAIWNYIHCMFGIRYDDYDYGEINQLLDRSFKVYIKTVVCTPEKVTKRMYDSFWRQFKHSEKVHVNLLLIEARMQAELLYALRAITRYMT

SEQ ID NO: 2 - protein, 492 aa, Mus musculus
MNRGGSSASASANYLLCTNCRKVLRKDKRIRVSQPLTRGPSAFIPEKEVVQANTADERTNFLVEEYSTSGRLDNITQVMSLHTQYLESFLRSQFYMLRMDGPLPLPDRHYIAIMAAARHQCSYLINMHVDEFLKTGGIAEWLNGLEYVPQRLRNLNEINKLLAHRPWLITKEHIQKLVKTGENNWSLPELVHAVVLLAHYHALASFVFGSGINPERDPGIANGFRLISVSSFCVCDLANDNSIENTSLAGSNFGIVDSLGELEALMERMKRLQEDREDDETTREEMTTRFEKEKKESLFVVPGETLHAFPHSDFEDDVIVTADVSRYIEDPSFGYEDFARRGEEHLPTFRAQDYTWENHGFSLVNRLYSDIGHLLDEKFRMVYNLTYNTMATHEDVDTTTLRRALFNYVHCMFGIRYDDYDYGEVNQLLERSLKVYIKTVTCYPERTTKRMYDSYWRQFTHSEKVHVNLLLMEARMQAELLYALRAITRHLT

SEQ ID NO: 3 - protein, 480 aa, Mus musculus
MIVADSECHSEIKGYLPFTRGGVAGPETREEHREGQARRGSRGPSAFIPVEEILREGAESLEQHLGLEALMSSGRVDNLAVVMGLHPDYLSSFWRLHYLLLHTDGPLASSWRHYIAIMAAARHQCSYLVGSHMTEFLQTGGDPEWLLGLHRAPEKLRKLSEVNKLLAHRPWLITKEHIQALLKTGEHSWSLAELIQALVLLTHCHSLASFVFGCGILPEGDAEGSPASQAPSPPSEQGTPPSGDPLNNSGGFEAARDVEALMERMRQLQESLLRDEGASQEEMENRFELEKSESLLVTPSADILEPSPHPDILCFVEDPAFGYEDFTRRGTQAPPTFRAQDYTWEDHGYSLIQRLYPEGGQLLDEKFQVACSLTYNTIAMHSGVDTSMLRRAIWNYIHCVFGIRYDDYDYGEVNQLLERNLKIYIKTVACYPEKTTRRMYNLFWRHFRHSEKVHVNLLLLEARMQAALLYALRAITRYMT

SEQ ID NO: 4 - DNA, 48 bp, Artificial
  primer
ctactactactaggccacgcgtcgactagtacgggnngggnngggnng

SEQ ID NO: 5 - DNA, 22 bp, Artificial
  primer
gccagggttttcccagtcacga

SEQ ID NO: 6 - DNA, 32 bp, Artificial
  primer
ctactactactaggccacgcgtcgactagtac

SEQ ID NO: 7 - DNA, 38 bp, Artificial
  primer
tgtaaaacgacggccagtgtgaaggctgtgcgaggccg

SEQ ID NO: 8 - DNA, 21 bp, Artificial
  primer
cctggaacggaacctcaaaat

SEQ ID NO: 9 - DNA, 20 bp, Artificial
  primer
gggcttcaaggagcagcaag

SEQ ID NO: 10 - DNA, 8950 bp, Artificial
  gene trap vector pTbetageo
tgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggaattcatgggaagaggaaccgaaagtatgtttttcagatgttctttctcagaaataggagtttgcggaggttggagtgtgtgttgtaggacacgaaccccagggtggaggagactggaggacagagccctctttcccagggagggaaggaggagagtttgagatccgctccggaagtcggggttcaggtttgagcaggccaggcctctcccgtggtctcgccctcttgtcctagaagcctcactggccaggtgtaagccaggtcgtgggtgccgagccctgctccctcatcctcagcatggatgtgaagaggactgtatggcgtgcgggtgtgtgtgaccgtgggtacacttaaaacaccgggttttggatctgcactgtcccggatgtcctctggtgctcaaagacccttttgggtttgccctttggtaagagcgccgggatctacttgtctggaggccagggagtcctcagccgaggcttgccgcccctgactgcactgcactgagtagtggatgggagagtctggtaccgcactgccggtttcctccaccatccccgcagcgcagggcagtgcattccgtcctggctgcgaagggggatggtcgggccttctccagcctcttccgcttctagcggaaggggccttgatggaagggcccgcatgtctccaaagttgattcatgcttcttgcacagagaaagaccagaaagaaggtctcaagttttagccggtagcccggatggcctttcctgcacggcaccatatgaaccttgtgaccctgacaaagagacccctctaacccaagacccctaccacttaccctttccctttgaaggctttcccacaccaccctccacacttgccccaaacactgccaactatgtaggaggaaggggttgggactaacagaagaacccgttgtggggaagctgttgggagggtcactttatgttcttgcccaaggtcagttgggtggcctgcttctgatgaggtggtcccaaggtctggggtagaaggtgagagggacaggccaccaaggtcagccccccccccctatcccataggagccaggtccctctcctggacaggaagactgaaggggagatgccagagactcagtgaagcctggggtaccctattggagtccttcaaggaaacaaacttggcctcaccaggcctcagccttggctcctcctgggaactctactgcccttgggatccccttgtagttgtgggttacataggaagggggacgggattccccttgactggctagcctactcttttcttcagtcttctccatctcctctcacctgtctctcgaccctttccctaggatagacttggaaaaagataaggggagaaaacaaatgcaaacgaggccagaaagattttggctgggcattccttccgctagcttttattgggatcccctagtttgtgataggccttttagctacatctgccaatccatctcattttcacacacacacacaccactttccttctggtcagtgggcacatgtccagcctcaagtttatatcaccacccccaatgcccaacacttgtatggccttgggcgggtcatccccccccccacccccagtatctgcaacctcaagctagcttgggtgcgttggttgtggataagtagctagactccagcaaccagtaacctctgccctttctcctccatgacaaccaggtcccaggtcccgaaaaccaaagaagaagaaccctaacaaagaggacaagcggcctcgcacagccttcactgctgagcagctccagaggctcaaggctgagtttcagaccaacaggtacctgacagagcagcggcgccagagtctggcacaggagctcggtacccggaagatctggactctagaggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaggggatcccccgggctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccccggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgggggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgac

SEQ ID NO: 11 - DNA, 20 bp, Artificial
  primer
cagccttgagcctctggagc

SEQ ID NO: 12 - DNA, 21 bp, Artificial
  primer
ctaccctgagaagacgacccg

SEQ ID NO: 13 - DNA, 21 bp, Artificial
  primer
ctaccctgagaagacgacccg

SEQ ID NO: 14 - DNA, 30 bp, Artificial
  primer
ggacaaatcaaggttacacagaaaaaagtc

SEQ ID NO: 15 - DNA, 20 bp, Artificial
  primer
cagccttgagcctctggagc

SEQ ID NO: 16 - DNA, 4520 bp, Artificial
  gene trap vector U3Cre
aatgaaagaccccacctgtaggtttggcaagctagcatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggctagcttaagtaacgccattttgcaaggcatggaaaaatacataactgagaatagagaagttcagatcaaggtcaggaacagatggaacagctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaattgcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagggatccatcgtagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggctagcttaagtaacgccattttgcaaggcatggaaaaatacataactgagaatagagaagttcagatcaaggtcaggaacagatggaacagctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 17 - DNA, 23 bp, Artificial
  primer
ccaatcttgcttctttgctgagc

SEQ ID NO: 18 - DNA, 22 bp, Artificial
  primer
ggctcatgcttgaatgtttcag

SEQ ID NO: 19 - DNA, 23 bp, Artificial
  primer
ccaatcttgcttctttgctgagc

SEQ ID NO: 20 - DNA, 20 bp, Artificial
  primer
atcatgcaagctggtggctg

SEQ ID NO: 21 - DNA, 21 bp, Artificial
  primer
ctaccctgagaagacgacccg

SEQ ID NO: 22 - DNA, 30 bp, Artificial; identical to SEQ ID NO: 14
  primer

SEQ ID NO: 23 - DNA, 21 bp, Artificial
  primer
ctaccctgagaagacgacccg

SEQ ID NO: 24 - DNA, 20 bp, Artificial
  primer
cagccttgagcctctggagc